(19) United States
(12) Patent Application Publication
(10) Pub. No.: US 2006/0141617 A1
(43) Pub. Date: Jun. 29, 2006

(54) Multilayered microcultures

(75) Inventors: Tejal Ashwin Desai, Wellesley, MA (US); Wei Tan, Chicago, IL (US)

Correspondence Address: GREENLEE WINNER AND SULLIVAN P C, 4875 PEARL EAST CIRCLE, SUITE 200, BOULDER, CO, 80301, US

(73) Assignee: THE BOARD OF TRUSTEES OF THE UNIVERSITY OF ILLINOIS, Urbana, IL (US)

(21) Appl. No.: 10/535,588

(22) PCT Filed: Nov. 19, 2003
(86) PCT No.: PCT/US03/36958

§ 371 Date: Dec. 12, 2005

Related U.S. Application Data
(60) Provisional application No. 60/427,646, filed on Nov. 19, 2002.

(51) Int. Cl. C12N 5/08; C12N 5/02

(52) U.S. Cl. 435/325; 435/366; 435/289.1

(57) ABSTRACT

A multilayer microculture capable of modeling complex in vitro structures such as mammalian tissues and organ structures is provided, along with methods for producing such a microculture and methods of using such microcultures for assaying for modulators of cell-cell interaction, cell migration, cell proliferation, cell adhesion or cellular or organismal physiology. Further provided are methods of identifying hazardous materials such as environmental toxins and pollutants (e.g., carcinogenic compounds), and methods of monitoring organismal physiology.

We claim the benefit of priority to U.S. Provisional Patent Application No. 60/427,646, filed Nov. 19, 2002, which is incorporated herein by reference in its entirety.

Support for the work provided herein was provided, in part, by NSF Career: #2528989 CRB. #t2245876. The federal government may have certain rights in this invention.

### FIELD OF THE INVENTION

The present invention relates generally to the field of cell maintenance and growth, cell culture, and tissue modeling.

### BACKGROUND

Biological cells are considered to be the fundamental unit of life, notwithstanding the debate over the status of viruses. Although fundamental, cells are quite complex, occasionally providing all of the structure and function necessary to support life, as in single-celled organisms. The situation grows even more complex when attempting to understand multi-celled organisms. In addition to studies designed to reveal the workings of individual cells, multi-cellular organisms present the challenge of mastering higher order processes involving cell-cell interactions, both direct and indirect, and the organization and functioning of a multitude of cells, both like and unlike, in higher order structures such as tissues and organs. To date, investigations into the structure and function of isolated cells have outpaced the more complex inquiries into the higher order structures of tissues and organs.

The relative lack of understanding of tissues and organs has not been for lack of interest. Many diseases and disorders that plague man, other animals, and plants have been associated with defects or abnormalities associated with tissues, or organs. The tremendous benefits attending an increased understanding of cellular activities within the context of a tissue or organ environment are apparent in terms of improved human and non-human animal health, the lowered health care costs associated therewith, and effective gains in agricultural yields. Notwithstanding these benefits, the inaccessibility of tissues and organs, particularly in humans, has limited the potential of in vivo investigations.

The majority of the ex vivo and in vitro studies of higher order organizations of cells has been accomplished by classical organ, tissue, and cell culture approaches. For example, cultures have been developed to sustains a variety of cells, including prokaryotic cells, eukaryotic plant cells, and eukaryotic animal cells, including human cells. These techniques have been extended to mixed cultures, in which more than one cell type is found, and even to multi-layered cultures having one or more cell types per layer. Additionally, ex vivo tissue culturing techniques have advanced. Common to all of these approaches is their macroscale orientation. Potentially significant, yet subtle, cellular interactions that contribute to, e.g., tissue structure and function, are not revealed by these approaches. Thus, a need exists for studying cell-cell behavior on a microscale.

In vivo, however, tissues exhibit a heterogeneous composition and a well-organized three-dimensional (3-D) structure. The tissues are composed of layers, membranes, tubes, and channels. Each function requires the appropriate components, cells, materials and signaling molecules, with sizes of organizational units typically in the micro-(10⁻⁶ meters) or nano-(10⁻⁹ meters) scale. Through the organization of these heterogeneous components into specific structures, sophisticated functions can be achieved. For example, the hierarchical vascular system of many multi-celled animals, including man, functions like a coordinated system of pipes and tubing for the passage of blood, or lymph. The vessels of the vascular system have three lamellar layers: adventitia, media and intima. The three layers, though only exhibiting thicknesses of about one hundred microns, are diversified in cell types, extracellular matrix compositions, and functional properties.

Recently, some attention has been paid to individual cell-cell behavior at the microscale level. One promising avenue has been the development of liquid cultures in microdevices generated using photolithographic techniques. Both individual-cell-type and mixed-cell-type microcultures have been reported. Liquid cultures, however, do not provide a good model for the in vivo environment of the vast majority of cells, an exception being those cells of eukaryotic animals that are found freely transported in such bodily fluids as blood and lymph. Notwithstanding these exceptions, however, liquid cultures frequently sacrifice the in vivo context for accessibility.

Quite recently, however, reports have surfaced regarding the use of constraining gels to culture cells on a microscale. These cultures, however, have been directed towards an investigation of the two-dimensional micropatterning of cells [Chen et al., Biotechnol. Prog. 14(3), 356-63, 1998], the control of cell-cell interactions in a two-dimensional co-cultures [Bhatia et al., Biotech. Prog. 14, 378-87, 1998], and the influence of surface microtexturing on cell contact guidance, again for a two-dimensional culture [Den Braber et al., J. Biomed. Mat. Res. 40, 291-300, 1998]; George Whitesides and colleagues nave also investigated the patterned culturing of cells, including a characterization of the in vitro propagation of a virus through such a cell culture [Endler et al. Biotechnol. & Bioengineer. 81, 719-725, 2003]. In each of these cases, an improvement in cell function over randomly oriented cell cultures was found. The single-layer, or two-dimensional, microcultures are incapable of realistically mimicking the cellular heterogeneity and three-dimensional microarchitecture found in the natural in vivo environment, however. That inability necessarily sacrifices the three-dimensional, ordered, cellular context in which most cells of a multi-cellular organism naturally exist.

One of the challenges for the development of in vitro tissue models or neotissues (tissue-engineered constructs), or for in vitro drug screening models attempting to capture effects not seen at the single-cell level, is the engineering of cell and matrix structures that better mimic the complex tissue microarchitecture found in vivo. Microtechnology, a collective term of micromachining, surface engineering and soft lithography, has achieved some features that mimic the in vivo structures on size scales relevant to living systems, i.e., from microns to nanometers. Previous work has demonstrated 2-D patterns of single cells and of two cell types on a solid surface.

Thus, a need continues to exist in the art for a flexible, multilayered approach to cell culture on a microscale that accommodates the types of cell-cell behavior more closely mimicking the in vivo environment of such cells, including the overall shape of a given microenvironment and the orientation of cells found therein. The need extends to multilayered microcultures that will facilitate the use of an array of analytical tools and techniques to be brought to bear on microcultures mimicking the tissues of multi-cellular organisms, such as man. That need extends to multilayered microcultures capable of modeling the tissues of organisms, as well as methods for furthering our understanding of the structure and function of tissues, and methods to identify modulators to correct tissue structure and/or function that is at risk of developing, or has developed, an abnormality such as a disorder or disease.

### SUMMARY

The present invention addresses the aforementioned need by providing a flexible and cost-effective approach to multilayered microculturing of cells that provides a more accurate mimic of in vivo tissues than approaches known in the art. The microculturing approach is flexible in being readily adapted to the culturing of multiple layers of a single cell type, to the culturing of multiple cell types in individual layers of a microculture, and to the culturing of mixed cell populations in one or more layers of a microculture. Further, each layer of these microcultures contains, in addition to cells, a biopolymer capable of polymerizing to provide a three-dimensional architectural framework for cell culture that approaches the in vivo microarchitecture of most cells of multicellular organisms. Moreover, these capabilities are realized at a microscale, thereby allowing for a wide array of precise investigatory tools to be brought to bear in monitoring such cultures. Armed with this technology, a variety of applications are apparent, including methods of generating neotissues, methods of monitoring the physiology of cultures, including cultures mimicking tissues, methods for identifying modulators of a variety of cellular behaviors, including cell-cell interaction, cell viability, cell proliferation, cell migration, cell adhesion and cell patterning.

One aspect of the invention provides a multilayer microculture comprising a plurality of three-dimensional non-fluid layers, wherein each layer comprises at least one cell type and a biopolymer selected from the group consisting of collagen, chitosan; fibronectin, matrigel, fibrin, and mixtures thereof, and wherein each layer comprises a width less than one millimeter. In some embodiments, each layer comprises a distinct cell type; in other embodiments, a mixture of cell types is found in at least one layer. In some embodiments, at least one layer is attached to an optically translucent, and preferably optically transparent, substrate or support. An exemplary support is a glass slide, preferably derivatized with amino groups (e.g., by reaction with 3-aminopropyltriethoxysilane). Also preferred is a glass slide derivatized by addition of amino groups and subsequent reaction with an aldehyde group (e.g., a cross-linker) to generate a glass support derivatized to yield a reactive aldehyde that can form covalent imino groups with the amines of a protein. Yet other embodiments exhibit at least one layer attached to a support that is effectively transparent to some range of the electromagnetic spectrum useful in monitoring the microsystem. In preferred embodiments, the layers comprise a first layer that is immobilized and wherein the first layer is resistant to a shear force associated with a 5 μl/min lateral flow of a cell-biopolymer fluid across the face of the first layer. In some embodiments, the above-described microculture mimics a mammalian tissue, e.g., in terms of development and/or physiology.

In another aspect, the invention provides a method for producing a multilayer microculture comprising (a) introducing a first material comprising a first cell matrix compound and a first cell type to a microstructure by microfluidic delivery, wherein the material is introduced as a fluid; (b) attaching the first material to at least one surface of the microstructure; (c) incubating the first material under conditions suitable for at least one component of the material to polymerize and for the material to contract in at least one dimension; (d) repeating step (a) with a second material comprising a second cell matrix compound and a second cell type; (e) attaching the second material to the first material; and (f) incubating the second material under conditions suitable for at least one component of the second material to polymerize, thereby producing a multilayer microculture. Methods according to this aspect of the invention include a first material and/or a second material (and any additional materials, e.g., a third material, as will be apparent below) further comprising a cell culture medium. The conditions suitable for polymerization and contraction include time and temperature. In some embodiments, the first and second cell types are identical. The invention also comprehends embodiments in which the first cell matrix compound and the second cell matrix compound are the same. Embodiments of this aspect of the invention also include methods further comprising preparing a third layer of microculture by incubating the second material under conditions suitable for the second material to contract; and repeating the steps described above for preparation of a second layer, thereby producing a three-layer microculture. Of course, the invention generally comprehends methods of preparing microcultures comprising a plurality of layers. Further, the methods described herein can be practiced with any of the microstructures described above, such as microstructures comprising a glass or a derivatized glass support.

Embodiments of this aspect of the invention also include methods as described above, wherein the microstructure comprises a plurality of microchannels and at least one microfluidic aperture.

Another aspect of the invention is drawn to a method of screening for a biohazardous material comprising (a) incorporating a test material into at least one layer of a multilayer microculture as described above; (b) incubating the microculture; and (c) measuring culture development in the presence of the test material relative to the culture development in the absence of the test material, wherein a difference in response relative to a microculture lacking said test material identifies a biohazardous material. Any property or characteristic of culture development known in the art or disclosed herein may be subject to measurement, including, but not limited to, cell viability, cell proliferation, cell migration, cell adhesion, or cell patterning (e.g., spatial patterning) wherein the cell comprises at least one cell type of the microculture, and extracellular signaling.

Yet another aspect of the invention provides a method for monitoring physiological health comprising (a) obtaining a biological sample from a mammalian subject; (b) incorporating the biological sample into at least one layer of a multilayer microculture as described herein; (c) incubating the microculture; and (d) measuring culture development in the presence of the biological sample relative to the culture development in the absence of the biological sample, wherein a difference in response relative to a microculture lacking the biological sample provides an indication of the physiological health of the subject. Again, any property or characteristic of culture development known in the art or disclosed herein may be subject to measurement, including, but not limited to, the viability, proliferation, migration, adhesion, or patterning of at least one cell type, and extracellular signaling. A preferred subject according to this aspect of the invention is a human, although domesticated farm animals, pets, and other mammals are also contemplated.

Still another aspect of the invention comprehends a method for identifying a modulator of tissue development comprising (a) incorporating a candidate modulator of tissue development into at least one layer of a microculture as described above; (b) incubating the microculture; and (c) measuring the tissue development in the presence of the candidate modulator relative to the tissue development in the absence of the candidate modulator, wherein a difference in response relative to a microculture lacking the candidate modulator identifies a modulator of tissue development. Viability, proliferation, migration, adhesion, and spatial patterning of at least one cell type of the microculture are exemplary indicators of tissue development suitable for measurement according to this aspect of the invention.

Yet another aspect of the invention is a method for identifying a modulator of cell-cell interaction comprising (a) incorporating a candidate modulator of cell-cell interaction into at least one layer of a microculture as described above; (b) incubating the microculture; and (c) measuring cell-cell interaction in the presence of the candidate modulator relative to cell-cell interaction in the absence of the candidate modulator, wherein a difference in response relative to a microculture lacking the candidate modulator identifies a modulator of cell-cell interaction. In some embodiments of this aspect of the invention, the microculture containing the candidate modulator and the microculture lacking the candidate modulator are the same, although the candidate modulator is only added to a subset of layers of such a microculture. Although those embodiments provide advantages in terms of reduced cost and ease of use, the embodiments are best-suited to methods involving regular, or continuous, measuring, to ensure that the progressive effects of a diffusing candidate modulator do not confound the results.

Another aspect of the invention is a method for identifying a modulator of cell migration comprising (a) incorporating a candidate modulator of cell migration into at least one layer of a microculture as described above; (b) incubating the microculture; and (c) measuring cell migration in the presence of the candidate modulator relative to cell migration in the absence of the candidate modulator, wherein a difference in response relative to a microculture lacking the candidate modulator identifies a modulator of cell migration.

Still another aspect of the invention is drawn to a method for identifying a modulator of cell proliferation comprising (a) incorporating a candidate modulator of cell proliferation into at least one layer of a microculture as described above; (b) incubating the microculture; and (c) measuring cell proliferation in the presence of the candidate modulator relative to cell proliferation in the absence of the candidate modulator, wherein a difference in response relative to a microculture lacking the candidate modulator identifies a modulator of cell proliferation.

Yet another aspect of the invention is a method for identifying a modulator of cell adhesion comprising (a) incorporating a candidate modulator of cell adhesion into at least one layer of a microculture as described above; (b) incubating the microculture; and (c) measuring cell adhesion in the presence of the candidate modulator relative to cell adhesion in the absence of the candidate modulator, wherein a difference in response relative to a microculture lacking the candidate modulator identifies a modulator of cell adhesion.

The above-described aspects of the invention, drawn to methods of identifying a modulator of cell migration, cell proliferation, or cell adhesion, are each also amenable to embodiments in which the microcultures containing, and lacking, the candidate modulator are the same. In some embodiments of each of these aspects of the invention, the microculture containing the candidate modulator and the microculture lacking the candidate modulator are the same, although the candidate modulator is only added to a subset of layers of such a microculture. As noted above, although these embodiments provide advantages in terms of reduced cost and ease of use, the embodiments are best-suited to methods involving regular, or continuous, measuring, to ensure that the progressive effects of a diffusing candidate modulator do not confound the results.

The invention further comprehends a kit for performing any of the above-described methods comprising a multilayer microculture comprising a plurality of three-dimensional non-fluid layers, wherein each layer comprises at least one cell type and a biopolymer selected from the group consisting of collagen, chitosan, fibronectin, matrigel, fibrin, and mixtures thereof, and wherein each layer comprises a width less than one millimeter, and package instructions for using the contents of the kit to perform the relevant method.

Numerous additional aspects and advantages of the invention will become apparent to those skilled in the art upon consideration of the following detailed description of the invention, which describes presently preferred embodiments thereof.

### BRIEF DESCRIPTION OF THE DRAWING

FIG. 1. Surface modification procedure involved in cell-collagen gel immobilization.

FIG. 2. Flow chart of preparing cell patterns on the substrate using microfabrication and microfluidic techniques.

FIG. 3. The contraction of collagen matrix (0.8 mg/ml) with cell density of 3×10⁵cells/ml.

FIG. 4. Schematic illustration of the approach using microfluidics to create 3D hierarchical system for 3-layers of cells and biopolymer matrices.

FIG. 5. Time-lapse video image sequences of the bottom layer under shear stress of fluidic delivery of a new layer.

FIG. 6. 3-D structure images demonstrate multilayer cells.

FIG. 7. SEM micrographs show the ultrastructure of different matrices.

FIG. 8. SEM micrograph of the two-layer structure of the model.

FIG. 9. Biomimetic cellular interaction paradigm.

FIG. 10. Schematic illustration of using the bio-mimetic layer structures for drug screening model.

FIG. 11. SMCs cultured on top of fibroblasts-collagen layer.

FIG. 12. SEM picture of SMC-matrigel cultured on top of fibroblasts-collagen.

FIG. 13. HUVECs migration on the layer of SMC-matrices.

FIG. 14. ICAM-1 expressions in different co-cultures.

FIG. 15. Cytoskeleton structure in different settings of two-layered co-cultures of HUVECs and SMCs.

FIG. 16. A schematic diagram of microstructured cell and ECM (extracellular matrix) assembly.

FIG. 17. Microscopic evidence of capillary-like tissue formation by the HUVEC migration from HUVEC-ccf micropattern with layer of fibroblasts on top.

### DETAILED DESCRIPTION

The invention provides a versatile, microfluidics-based approach to the preparation of multilayered microcultures amenable to the in vitro realization of three-dimensional, multicellular microarchitectures that mimic natural tissues and are, therefore, suitable for use as neotissues. Further, these multicellular microarchitectures can be subjected to a variety of chemical (including biochemical), physical (including electromagnetic, eletromechanical, and mechanical) and biological influences to engineer modified architectures useful, e.g., in treating disease. Additionally, the multicellular microcultures are well suited as bases for assays to identify modulators of any of a variety of cell behaviors (e.g., cell-cell interaction, cell viability, cell proliferation, cell migration, cell adhesion, and, generally cellular patterning in multicellular organizations such as tissues) influenced by an extracellular stimulus. Such modulators are useful in treating a variety of disorders and diseases in mammals, including humans; the modulators are also useful in ameliorating the symptom of a mammalian disease or disorder.

The invention comprehends a wide variety of applications for the multilayered microcultures, including applications in basic cell biology, tissue engineering, drug discovery, and biosensors. Compared to current culturing techniques, the advantages of multilayered microculturing notably include compatibility with 3-D multilayer cell patterning versus the 2-D patterning characteristic of monolayer culturing. Microscale control can be achieved on the surface as well as the thickness (the third dimension). However, all current techniques are limited to providing microscale control of the culture surface. Additionally, the invention provides the advantage of being able to introduce biopolymer matrices using microscale cell/tissue engineering techniques. Further, the invention contributes to our understanding of cell-cell interactions. Details of the usefulness, and advantages, of the invention are elaborated below.

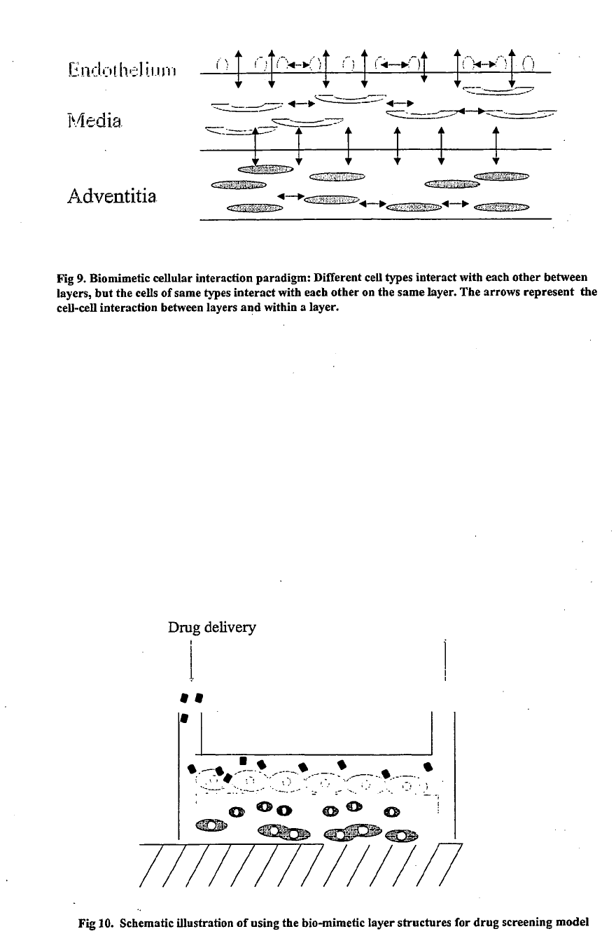

In one aspect, this invention is directed to a cell culture system. The well-controlled multilayer microstructures re-establish, or mimic, an in vivo-like cell-cell interaction paradigm for in vivo research. FIG. 9 shows the cell-cell interaction paradigm in the vascular system, as well as other laminar-structured systems in vivo. As shown in the figure, the paradigm for biomimetic cellular interaction is that, generally, cells of different types interact with each other between layers (or within layers, depending on design), while cells of the same type interact with each other within the same layer (or between layers, again depending on design). Therefore, an ideal in vitro model should incorporate a biomimetic pattern of heterotypic cell-cell interactions, and homotypic cell-cell interactions, in cell-matrix microenvironments. The invention provides such an ideal co-culture model that facilitates different types of interactions. Most co-culture models in use employ a random mode, while the rest of the known culture models have some kinds of control; none of them is able to control the 3-D co-cultured cells in microscale size.

In another aspect, this invention is directed to a microdevice used for cell-based assays for drug screening. Ultimately, any potential therapy must have efficacy at the cell, tissue, and human level. The use of cell-based assays is an important source of information driving this process. Cell functions are comprised of many interconnecting signaling and feedback pathways. Compound screens based on isolated targets or cell preparations cannot accommodate this complexity. Thus, a more complete understanding of compound effects requires testing whole living cells, co-cultures, or tissues. The microfluidic-based device of the invention, with cell-material multilayers inside the channels, may be designed to capture more insights into complex cellular activities than other commonly used cell-based assay formats. The three-dimensional cultures may be used in vitro to screen a wide variety of compounds for effectiveness as pharmaceutical agents, for cytotoxicity as pharmaceutical agents, for function as growth/regulatory factors, as anti-hypertensive agents, and the like. A possible format for the new cell-based assay is shown in FIG. 10. By using the vascular system inside the microchannel, the delivery of the drug in the channel is very similar to that in the blood vessel. The drug is transported by the fluid (analogous to blood) along the top confluent EC layer (analogous to vascular endothelium), with a controlled flow rate (analogous to physiological blood flow) by a syringe pump. Because a PDMS stamp has good optical properties for direct imaging, a possible detection method for monitoring cell structure and/or function is to use various florescence probes. For example, the activity of a cytotoxic compound can be measured by its ability to damage or kill cells in culture. This may readily be assessed by vital staining techniques. The effect of growth/regulatory factors may be assessed by analyzing the cellular content of the matrix, e.g., by total cell counts, and/or by differential cell counts. This may be accomplished using standard cytological and/or histological techniques including the use of immunocytochemical techniques employing antibodies that define type-specific cellular antigens. The potential advantages of this 3-D microculture, as opposed to conventional cell cultures or organ cultures, are numerous. For instance, when cells are placed in a defined ECM (extracellular matrix) environment like the multilayer patterns, fibroblast, SMC (smooth muscle cell) and EC (endothelial cell) differentiation close to the in vivo situation can be obtained, and cell functions and responses to pharmacological agents or active substances or products can be investigated at the neotissue level. In addition, many cellular or tissue activities are amenable to detection in the microdevice, including: diffusion rate of the drugs into the layered tissues in transported flow channel; cell morphology and differentiation changes at different layers; cell locomotion, apoptosis, and the like. Further, the effect of various drugs on different types of cells located at different layers of the three-dimensional system may be assessed.

In yet another aspect, this invention is directed to an in vitro model for studying inflammatory processes and healing responses. The microcirculation constitutes the functional interface between the circulating blood and the interstitial space. To gain access to sites of inflammation, leukocytes must pass the endothelial barrier. The recruitment paradigm encompasses leukocyte margination, capture, rolling, activation, firm adhesion, and transmigration. The conventional in vitro model for this type of research employs a membrane seeded with a confluent EC monolayer or a co-culture model with ECs and SMCs seeded on the opposite sides of a membrane. With or without the existence of cytokines, the interaction of inflammatory cells and ECs is examined. The invention provides an in vitro model that will be able to provide more insights in the inflammatory process and into the immune response because the complete supporting layers, besides a confluent EC monolayer, are provided. The embodiment is similar to FIG. 10, but substituting a leukocyte cell suspension for the drug solution.

In yet another aspect, this invention is directed to a method for generating a hierarchical, heterogeneous microstructure of hybrid biopolymer matrices as a 3-D scaffold. The scaffolds are used to research the building of hybrid matrices derived from different types of biopolymers with specific microarchitectures, and exploring the significance of this structure on cell adhesion and migration, cell-cell interaction, as well as “neotissue” formation. Microfabrication techniques offer the potential to modulate, on a cellular level, the biochemical composition as well as the topography of the substrate, the type of cell neighboring each cell, and the medium surrounding each cell. Using the materials and methods disclosed herein, the problems in adapting the biomaterials (e.g., cell, biopolymers) to a microscale format have been largely overcome. This invention provides ways to control the biomaterials at microscale-levels, so as to achieve greater control over scaffold configuration and materials. With or without the subsequent remodeling work of seeded cells, the fabricated hybrid biopolymer can be lyophilized/freeze-dried.

In yet another aspect, the invention is directed to a method for generating biomimetics that reproduce the microarchitecture of a tissue in vivo by arranging a suitable microenvironment(s) for cells. The microenvironment(s) for cells include material, cellular, and molecular environments. In vivo, the extracellular matrix serves as a reservoir for growth factors and other functional proteins, and the distribution of those factors or proteins is very different at various locations, and/or exhibits a concentration gradient at nearby locations. Multilayer structures of biomatrices can allow different growth factors to be added to different layers according to the needs of cells and their existence in vivo. Controlling the location of those factors or proteins may direct the process of specific cell remodeling and functioning, and also may provide the molecular cues to the whole system, resulting in such beneficial developments as neotissue, cell remodeling, and the like.

In yet another aspect, the invention is directed to a method for tissue-engineered products. The invention can be employed in engineering a variety of tissues including, but not limited to, the cardiovascular system, bones, teeth, and skins. Laminar tissue structures exist in all of these tissue types. The invention also provides a method of organogenesis comprising providing cells, mixing the cells with appropriate natural or synthetic biopolymers, delivering cell-matrices in specific ordered layers to form a “neotissue,” and growing the “neotissue” inside one or more channels of a microdevice to allow the formation of tissue. If the substrate and/or stamp are substituted by biocompatible materials, such as poly-lactide-co-glycolide acid (PLGA), the cells cultured in this manner may be used for transplantation or implantation in vivo. Moreover, the ability to spatially localize and control interactions of several cell types in polymeric materials presents an opportunity to engineer hierarchically and more physiologically correct tissue analogs. The arrangement of multiple cell types in two- and three-dimensional arrangements has beneficial effects on cell differentiation, maintenance, and functional longevity.

In yet another aspect, the invention is directed to a method for directed cell migration. For example, using an “out-channel” culture mode for co-culturing fibroblasts with HTVECs in collagen-chitosan-fibronectin matrices, fibroblasts on the top layer are found to direct the migration of ECs at the bottom, and sprout formation, like the beginning of the process of angiogenesis, is observed (Example 3).

In yet another aspect, the invention is directed to a method for generating a functional bioartificial vessel/capillary. Important steps have been made toward building bioartificial vessels (Example 1). The vascular network is far more complicated than a simple channel. It is actually a network of vascular channels. The combination of microfabrication techniques for surface patterns and microfluidic multilayer patterning, is expected to result in a more in vivo-like vascular network. The in vivo vascular system can provide guidance in designing masks for an in vitro system. The invention also provides the capacity to take a further step in bioartificial vessel/capillary engineering, providing the flexibility needed to accommodate preferred orientations of cells and matrices in a specific layer. The SMCs in vascular tissue in vivo have an orientation perpendicular to the orientation of endothelial cells. The cells in endothelium are oriented parallel to the vessel wall, while SMCs form a circumferential configuration around the wall in order to control the diameter of the vessel by contraction. Other electrical or mechanical components may be added to the microdevices and multilayer microcultures to align the fibers in biopolymer matrices. Cell orientation is closely related to the alignment of the matrix fibers due to contact guidance. For example, the alignment of collagen is found when a magnetic field or fluid forces are applied [Roy et al., Exp. Cell Res. 232, 106, 1997; Friedl et al., Cell. Mol. Life Sci. 57, 41, 2000]. Taking advantage of the different matrix components used in various layers, it is possible to apply magnetic or mechanical forces in a specific direction. If such forces can only affect the alignment of one type of matrix, cells in one layer could be selectively oriented if the cells are seeded in a matrix responsive to the exerted force. A bioartificial capillary is also useful in providing nutrients for other bioartificial organs, thus forming embedded capillaries inside some other tissue can be implemented by the addition of assembled matrices and cells (as other types of tissue) on top of the capillary-like patterned layer structure after removing stamps. Other important factors in bioartificial vessel design can also be accommodated. For example, shear stress from flow has proven to be essential to EC differentiation and function.

In yet another aspect, the invention is directed to a method for preparing a cell microarray. Cellular arrays or patterns may constitute the future “lab-on-a-chip.” These miniaturized cell cultures will facilitate the observation of cell dynamics with faster, less noisy assays, having built-in complexity that will allow cells to exhibit in vivo-like responses to the array. The invention also provides a method for maintaining pattern integrity in a cell array.

In yet another aspect, this invention is directed to a method for preparing a biological sensor. Cell-based biosensors can provide more information than other biosensors because cells often have multifaceted physiological responses to stimuli. Cells ranging from E. coli to cells of mammalian lines have been used as sensors for applications in environmental monitoring, toxin detection, and physiological monitoring [Pancrazio et al., Ann. Biomed. Eng. 27:697-711, 1999]. A corresponding handheld electronics system for sample analysis can be integrated into a microdevice comprising a multilayered microculture according to the invention.

In yet another aspect, the invention is directed to a method for understanding fundamental processes in cell biology and cell-matrix interaction. As illustrated in Example 1, the different matrices have different effects on cell migration. The in vivo remodeling process is a complicated, dynamic, reciprocal process between cells and matrices. The materials and methods of the invention are able to capture the complexity of these biological systems, rendering these systems amenable to investigation and beneficial manipulation.

Furthermore, coupled with imaging tools, such as optical coherence tomography (OCT), real-time analysis of cellular behavior in the multilayered microcultures is expected. Examples of cell and tissue studies amenable to real-time analysis include cell-cell interactions, dynamic 3-D engineered tissue construction and monitoring, structure-function investigations in tissue engineering, and the process of cell remodeling matrices in vitro.

The 3-D multilayer microsystem comprising a 3-D microstructure is created by a multistep process. The production of such a microstructure generally includes the following processes: (I) Modifying the surface of the substrate; (II) Microfabrication of PDMS (poly(dimethylsiloxane)) microdevices; (III) Preparing various reconstituted biopolymer matrices; (IV) Microfluidic delivery; (V) Contraction process of matrix by cells; (VI) Multilayer patterning; and (VII) Establishing a cell culture. A detailed description of each process is provided below.

Modifying the Surface of the Substrate

Chemical bonding protocols which alter the surface chemistry of silicone, glass, or other substrates will immobilize proteins applied to such derivatized structures (e.g., surfaces). Three-dimensional collagen, modified collagen matrices (e.g., collagen-chitosan), other biopolymer matrix materials (see below), or a cell-biopolymer (e.g., collagen) mixture is covalently bound to the silicone or glass surface by the following multistep process, as shown in FIG. 1. An APTES-GA-activated (3-aminopropyltriethoxysilane glutaraldehyde-activated) substrate surface was used. APTES-GA-activated surfaces have been used to immobilize antibodies or proteins [Yuan et al., Analyst, 126, 1456 (2001); Jonas et al., J. Biomed. Matls. A 57, 550-558, 2001], but never applied to the immobilization of 3-D biopolymer matrices. A “three-dimensional” structure (e.g., culture, matrix, or the like), as used herein, has a minimum dimension of 50 μm, and is distinct from two-dimensional cultures, matrices, and the like, notwithstanding any existing third dimension of relatively trivial magnitude (i.e., less than 50 μm) in such structures. This process has been derived from, but altered in several ways from, those published procedures.

The silicone or glass substrate is chemically functionalized with OH groups by piranha etching with a mixture of 3 parts of hydroperoxide (30% v/v) and 7 parts of concentrated sulfuric acid for 30 minutes. Slides are then rinsed thoroughly with pure water and blown dry with nitrogen. This treatment generates a hydrophilic surface with a water contact angle of less than 5°.

An amine group is attached to the surface via reaction with the vapor of 3-aminopropyltriethoxysilane (APTES) [FIG. 1(a)]. This is done by placing the substrate in a preheated vacuum oven, where a small dish loaded with 600 μl of APTES is also placed. A low vacuum is applied in the oven, as would be known in the art, and the surfaces are baked at 60° C. for 10 minutes to saturate the volume with APTES vapor, and then the heat is increased to 150° C. for 60 minutes to facilitate silanization of the surface. Silanization with APTES can either be done by incubating the substrate in a propanol solution of APTES or by vapor-depositing the silanes, as described here. For cell and fluorescence applications, the solution-based silanization generates much higher fluorescent backgrounds and lower cell viabilities, compared to the vapor-based silanization procedure.

An aldehyde cross-linker is attached to the amine group via reaction with 6% (v/v) glutaraldehyde (GA) in phosphate buffer for 10 minutes [FIG. 1(b)]. This yields an aldehyde that can form an imine linkage with a primary amine on a macromolecular chain of the biopolymer. Subsequently, a thorough rinse with a continuous water flow is used to remove residues of GA, preferably resulting in the complete removal of unreacted GA. Without proper removal, the residual GA may greatly influence the crosslinking of a gel matrix, cell viability, and fluorescent background. For the purpose of long-term cell culture, the substrate is sterilized in 70% ethanol for 20 minutes and dried in a conventional biohood. Finally, a poly(dimethylsiloxane) (PDMS) stamp is laid on the modified substrate with the microchannel structure facing down. The biopolymer that will form the matrix, e.g., pure collagen, modified collagen, other matrix materials as described herein, or mixtures thereof, is delivered into the microchannel by microfluidics. Although a collagen gel matrix is a large structural polymer, collagen is made of polypeptide chains, which contain amino groups that are suitable for covalent linkages to a substrate. The collagen material is attached to the surface through the reaction between the amine group on the collagen and the aldehyde group on the surface [FIG. 1(c)].

Ellipsometry measurement of thin films on silicon substrates shows that the APTES film is about 3 nm thick and the GA layer is about 0.4 nm thick. The efficiency of the immobilization of cell-collagen matrix on the substrate (e.g., slide) is evaluated by the percentage of the cell-matrix micro-stripes left on the slides after peeling off and rinsing with buffers. This value is the retention %. The APTES-GA-coated surfaces have much higher retention (>90%) compared to other tested surfaces, such as fibronectin-coated substrates, APTES-coated substrates, and uncoated substrates, all of which have retentions as low as 20%. This indicates that the cell-matrix is strongly immobilized on an APTES-GA surface. Because the cell-matrix is a 3-D structure, the described immobilization process is different from protein or biomolecular immobilization approaches commonly used. The immobilization of a cell-matrix may occur through the reaction between APTES-GA on the surface and a part of a biopolymer chain near the surface, so that the retention percentage is lower than 100%. The immobilization is important for building multilayer structures inside channels by microfluidics, as well as for facilitating the characterization process of the microstructure after removing the stamp (e.g., PDMS stamp). Without immobilization, it is hard to control the multilayer structure because the existing layer, if not anchored, will drift in the flow. Also, in subsequent characterization and use of the immobilized cell-matrix, multiple washes and o'their processes are applied. For non-covalent binding of a cell-matrix, it is very easy to lose the features on the substrate. The coated substrates are evaluated by measuring cell viability. There is no significant difference in the viability of fibroblasts on fibronectin-, APTES-, or APTES-GA-coated glass, in comparison to the viability of fibroblasts on uncoated glass. In all cases, viability is higher than 85%. This suggests that the modified substrates have very good biocompatibility and are suitable for cell culturing.

Microfabrication of PDMS Microdevices

The original design of patterns on the film is generated using techniques known in the art of soft photolithography, such as the use of AUTOCAD 2000, with printing as a high-resolution plot (5000 dpi, Graphic Resources, Chicago, Ill.). The patterns are simply an array of channels with typical dimensions of 1 cm length and a width of 250 μm, 350 μm or 500 μm. The inlet and outlet are in direct or indirect fluid communication with the channels and are ladder-shaped, facilitating fluid flow into the channel or channels. Because the SU-8 photoresist used in photolithography is a type of negative photoresist, the channel area in the mask is transparent while other areas are totally black and will finally be removed. According to work described previously [Kane et al., Biomaterials 20, 2363 (1999)], channel sizes above 200 μm are preferred to pattern cells. With the addition of the matrix, the viscosity of the injected solution is increased, necessitating larger channel sizes. The mask film is attached to a transparent glass plate as a substitute for a photomask. Although the resolution of the mask film is lower than a photomask, mask film has the advantages of convenience and short preparation time, thus allowing one to try different designs with ease.

A new product in the SU-8 series, SU-8 250 (Microlithography Chemical Co.), was used. Our protocol could reliably produce 300 μm-thick films, which are sufficient for 3-D cell-matrix delivery. Silicon wafers, p60 mm, were cleaned with Soma clean at 40° C. in an ultrasonic water bath for 15 minutes, followed by rinsing with DI water and drying with nitrogen. Wafers were then put onto a 200° C. hot plate for 1 hour for dehydration. After cooling down from the baling temperature, a quantity of photoresist equivalent to a ˜4 cm radius area was dispensed under static conditions, allowed to spread for 25 seconds at 650 rpm, and then spun for 15 seconds at 850 rpm. The wafers were then soft-baked at 95° C. for greater than 6 hours (always overnight) on a horizontal hot plate. This was followed by exposing wafers with SU-8 resist on top seven times for 11 seconds each with collimated UV light of 20 J/cm² strength, and with 30 second intervals between each exposure time to avoid overheating the photoresist surface. Wafers were hard-baked at 95° C. for 15 minutes and developed in SU-8 developer for one hour with constant shaking. Fresh SU-0 developer was used instead of IPA to rinse the surface three times before drying with nitrogen. This approach generated less residual material on the surface. No post-bake process was performed in order to avoid deformation of the features. Finally, to prevent adhesion of the PDMS, about 150 Å of Chromium was coated onto the wafer using an E-beam varian evaporation system. Optical profilometry (Veeco NT3300) was used to characterize the width and height of the microchannels.

Small biomedical silicon tubing with a 0.3 mm inner diameter (Physiology Research Supplies) was glued to both the inlet and outlet pad of the channel. PDMS replications were prepared as previously described [Folch et al., J. Biomechl. Eizg. 121, 28 (1999)]. PDMS precursor and curing agent (Dow Corning (Sylgard 184)) were mixed in a proportion of 10:1, poured over the master in a container forming an approximately 7-mm-thick layer, put under low vacuum (approximately, 15-20 psi) to evacuate the bubbles from the microtrenches, and cured at 65° C. for 12 hours (8-hour overcure to avoid toxic bleaching). Breaking the vacuum periodically (about once per minute for a 5-6 minute period) was necessary to pop the bubbles on the surface, which in turn allowed bubbles at lower depths to reach the surface. The cured PDMS replica, constituting a microfluidic network of microchannels, was gently peeled off, always in a direction parallel to the trenches. The outlet tubing should be removed to reduce the pressure needed for fluidic delivery. Then replica stamps were autoclaved at 121° C. for 20 minutes, as would be known in the art. Coating of the channels with 1% bovine serum albumin (BSA) in PBS prevented cell attachment and matrix adhesion to the wall. BSA is a protein used commonly for rendering surfaces non-adhesive to cells. PDMS replicas could be reused after cleaning thoroughly. The surface properties of reused PDMS and fresh ones were compared in terms of their contact angles and BSA attachment, and no significant differences were found. It is essential to always keep the PDMS surfaces clean for tight sealing.

Preparing Various Reconsituted Biopolymer Matrices

Different types of biopolymer matrices can be used in generating multilayer structures. The applicable biopolymer matrices include, but are not limited to, collagen, modified collagen (e.g., collagen-chitosan, collagen-chitosan-fibronectin), matrigel, fibrin, and the like. All of these biopolymers are maintained in a solution phase to serve as a carrier for cells by mixing with a cell suspension, and then polymerization is induced within the microstructure by conventional manipulations, such as changing a physical condition like the temperature, osmolarity, ion strength, or the like. Such polymerization processes should not affect cell viability. After polymerization, cells remain viable in the 3-D biopolymer matrices, adhering to their fiber networks.

In one embodiment, composite collagen-chitosan gel matrices were formed from collagen and varying amounts of chitosan. The preparation method for this copolymer was derived from the method for preparing a collagen gel. Type I rat-tail collagen was dissolved in 0.1M of acetic acid, with a final concentration of 2 mg/ml. Chitosan was made soluble in 0.1M acetic acid by stining for one day. When reconstituting gel matrix, the pH and osmolarity of the solution are raised to physiological levels (pH 7.4; osmolarity 300 mosm) by gently and thoroughly mixing the following solutions in a microcentrifuge tube on ice: (1) 100 μl of 10× Hanks balanced salt solution; (2) a predetermined volume of collagen solution to obtain the desired final concentration in a final volume of 1 ml; (3) a predetermined volume of chitosan solution to obtain the desired proportion with collagen; (4) 7 μl 5% w/v NaHCO₃; (5) a predetermined amount of 1M NaOH so that the final solution pH is 7.4; and (6) pure water or complete medium to make the final solution volume equal to 1 ml. To avoid clumps in the solution, mixing was needed after adding the chitosan component. For preparing a cell-gel assembly, a concentrated cell suspension was added to partially substitute for the 6^{th} component (water or medium). The final solution volume equaled 0.7 ml, so 0.3 ml of cell suspension was added to make the final volume 1 ml. Collagen of 0.6 or 0.8 mg/ml and collagen-chitosan (1:1 w/w) of 0.6 or 0.8 mg/ml for each component was used, and the final cell concentration was 3×10⁵ cells/ml. The collagen-chitosan matrix preparation can be further modified by adding fibronectin solution (1 mg/ml, Sigma) in an appropriate relative quantity (1:1: 0.1 (w/w/w) collagen:chitosan:fibronectin) into the mixed collagen-chitosan solution before polymerization. Solutions were well-mixed and placed on ice until used. Matrigel can be polymerized directly from a purchased solution by increasing the temperature. Fibrin can be made by mixing fibrinogen and thrombin in appropriate proportions (100 mg/ml fibrinogen mixed with 500 IU/ml thrombin in the presence of 40 mmol/l calcium chloride) so that thrombin cleaves the fibrinogen to fibrin. Adjusting the temperature can also control this process, as would be known in the art (e.g., polymerization will occur at 37° C.). Because the polymerization of all these matrix materials is finally controlled by temperature, the prepolymer solution delivered into the channels by pressure-driven microfluidics should be kept at low temperature. In a multilayer microsystem, of course, the matrices may vary in the different layers.

Microfluidic

Conventional techniques for cell microfluidic patterning have been described previously [Folch, 1999]. Adapted procedures were used in this study to pattern the first layer of a multilayer cell-matrix assembly. For fluidics delivery via injection, a 1 cc syringe was used, but the needle was replaced with one of a smaller size (26GA) and longer tip.

To pattern cell-ECM (cell-extracellular matrix) assemblies, cell-matrix solutions were prepared, including cell-collagen, cell-collagen/chitosan or others, and injected through the closed microchannels formed by the PDMS stamp in contact with the modified glass/silicon substrate. The matrix solution has a higher viscosity than a corresponding cell suspension [Gerentes et al., Biomaterials 23, 1295 (2002); Ho et al., J. Contr. Rel. 77, 97 (2001)]; therefore, the required driven pressure was higher. The noncovalent binding between the PDMS stamp and the substrate may be affected by the high pressure, and should be monitored. To minimize the possibility of a leak, methods for decreasing the pressure are preferred, such as removing the outlet tubing. According to the fluidic equation: ΔP=12η LQ/wh³, where ΔP is pressure difference, η is viscosity, Q is volume flow rate, and L, w, and h are the dimensions of the channel, the requirement for channel sealing is higher due to the increase in pressure needed to transport the fluid. In our experimental set-up, it is difficult to pattern cell-matrix solutions with channel widths of less than 250 μm.

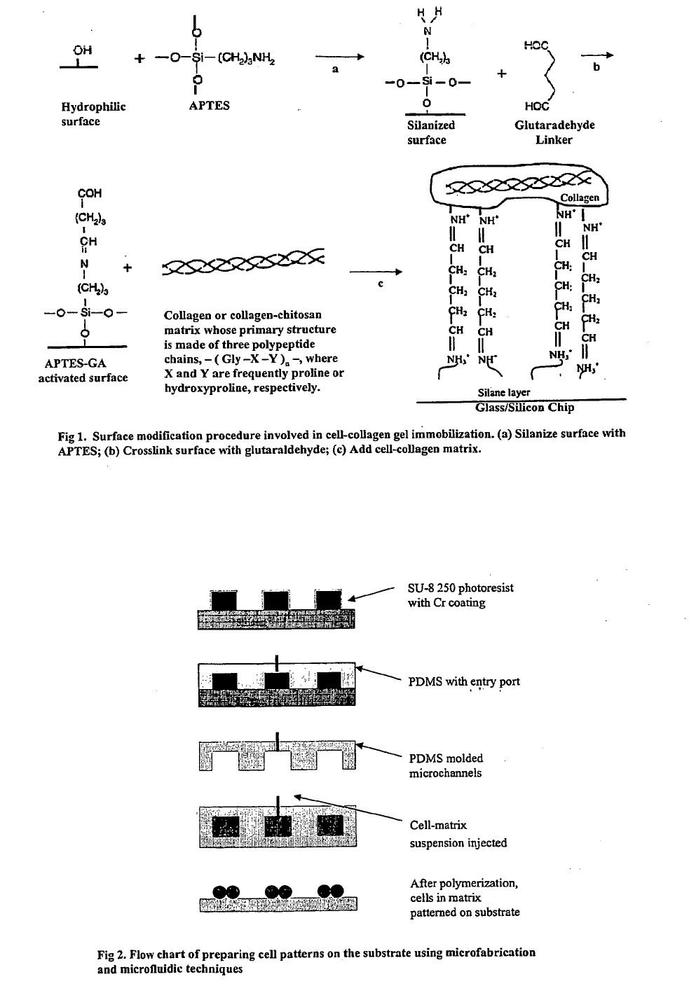

The polymerization time for the cell-ECM micropattern should be carefully controlled because the hydrated gel is easily dehydrated in the channel due to fast evaporation in the microchannel. Dehydration may lead to low viability of cells as well as the permanent deformation of biopolymer matrices. Polymerization times of about 15-20 minutes have been found to be optimal. In addition, a culture dish or other container in which the microdevice is being prepared may be filled with PBS (phosphate-buffered saline) to help prevent dehydration. Also, pressure on the stamp or other forces should be avoided to prevent the biopolymer matrix from collapsing or deforming. Finally, fresh culture medium is poured. FIG. 2 illustrates the overall process of cell patterning.

Contraction Process of Matrix Cells

One aspect of the invention is drawn to multilayered microcultures comprising at least one cell type, which may be a contractile or a non-contractile cell. In some embodiments, the cells of the microculture (i.e., at least one type of cell, but perhaps more than one type) are all non-contractile cells. Contractile cells, such as fibroblasts and smooth muscle cells, are able to exert traction forces on the matrix around them by deforming the matrix fibrils and causing the matrix to contract. Some other cells, such as the non-contractile endothelial cells, are able to secrete enzymes to degrade the matrix, which may also cause the size of the matrix to shrink. The process of matrix contraction by cells is used to construct a multilayered structure in microchannels. Following introduction of a given layer of cell-biopolymer matrix material, the layer becomes non-fluid by polymerization and contracts, thereby re-establishing a patent lumen in the channels. This lumen is available for introduction of another layer of the multilayer microculture.

Cell contraction is influenced by several parameters, such as cell type, cell concentration, matrix type, matrix composition, substrate surface chemistry, and time (FIG. 3). Thus, the thickness of each layer can be controlled at a microscale size. As shown in FIG. 3, if all the other parameters are fixed, the thickness of individual layers is dependent on the time of polymerization. The time-dependent contraction curve should be drawn for each specific cell type, specific cell concentration, and specific matrix composition, all of which are primarily determined by the biological requirements of a specific tissue layer. Generation of specific time-dependent contraction curves would involve no more than routine skill in the art and is within the skill of the ordinary artisan aware of the disclosure contained herein. In the working example described here, the initial cell concentration was fixed at 3×10⁵ cells/ml, collagen gel composition was fixed at 0.8 mg/ml, and the contraction was examined as a function of time. The ability of fibroblasts contracting a gel lattice has been studied over days and over hours. It was found that the matrix gel contraction exhibited a near-linear relationship with the hour, but did not change much after 24 hours in this condition [FIG. 3b]. The contraction of cells is very important in controlling the available spaces for building up the upper layers. Controlling the thickness of individual layers by the above-identified parameters affecting cell-matrix contraction makes this method more flexible and designable than macroscale cultures or than single-layer microscale cultures. For example, the invention contemplates a variety of substrate surface chemistries known in the art to immobilize at least a first layer of a multilayered microculture, with routine optimization procedures used to identify surface chemistries advantageously compatible with a desired degree of contraction.

Measurement of contraction of the matrices by cells was done by tracing the width of a cell-gel microstripe over time. Briefly, the PDMS stamp was placed onto an unmodified substrate and a cell-matrix fluid was delivered through the channels. After polymerization of the cell-matrix assembly inside the microchannel, the PDMS stamp was removed. The widths of micro-stripes of the cell-matrix on the glass slides were traced using a digital imaging microscope system at set times. Because the aspect ratio of the width and the height of the microchannel is near 1, the contraction of cell-matrix thickness (height) can be regarded as equal to the shrinkage in width. The contraction percentage was determined by the resulting microstripe width at certain times divided by the value of the original pattern size (i.e., the original width of the microstripe). The channel height for the 2^{nd} or 3^{rd} or subsequent fluid delivery (i.e., delivery of a second, third or subsequent layer of culture) can be estimated from the measurement of cell-matrix contraction:
h=h₀h_{g}
where h is the channel height change, which equals the original height (h₀) minus the existing gel height (h_{g}). For example, h₀ equals 300 μm, and h_{g} equals h₀ times the contraction percentage.
Multilayer Patterning

To seed a second cell type or even more types of cells after microfluidic patterning of the first layer, any of several approaches may be used. One approach is selective attachment. The patterned 3-D cell-matrices allow selective attachment of cells. For example, fibroblasts (approximately 1 million cells) are allowed to attach onto a HUVEC/matrix pattern on BSA-blocked substrates for about 20-40 minutes in PBS (shaking eliminates background attachment). The substrates are then rinsed and soaked in PBS for 10 minutes to remove non-adhered fibroblasts. Selective attachment can generate two-layer co-culture configurations, but has limitations: lack of precise selective attachment of the second cell type; only applicable to the co-culture of two cell types; only one matrix material, making it hard to control the third dimension; requirement for the matrix to contain highly cell-adhesive molecules; and no control over cell migration and the remodeling process.

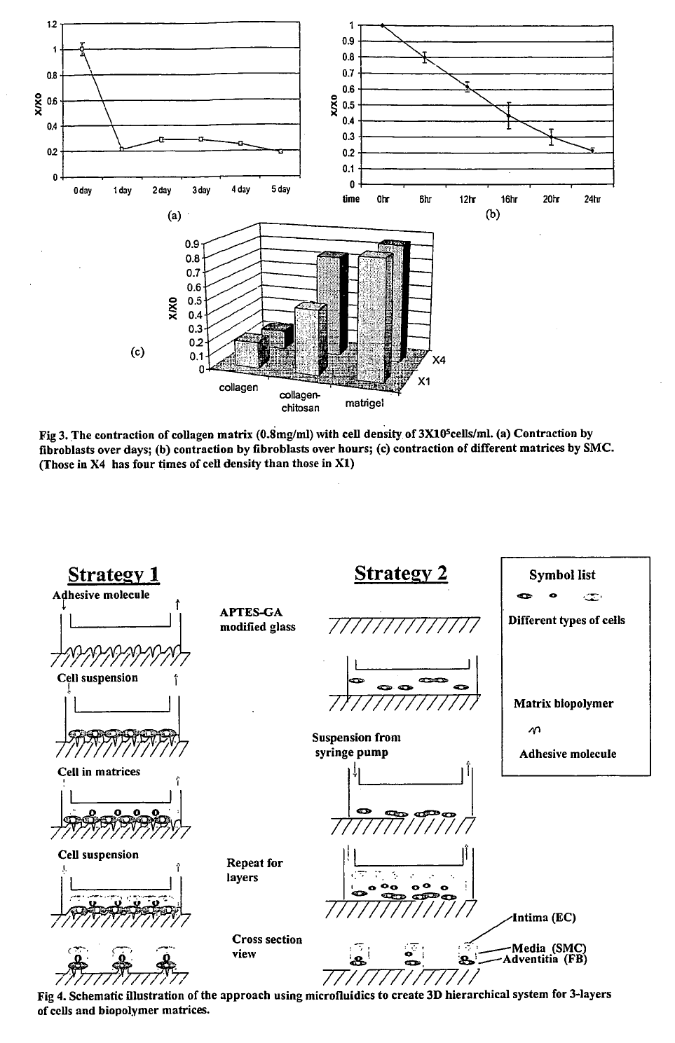

The strategies of multilayer patterning provide an alternative approach to the realization of 3-D multilayer microstructures. Suitable processes are illustrated in FIG. 4. These strategies overcome the shortcomings of selective attachment. There are at least two strategies designed for achieving the multiple layered configurations. The first strategy starts with patterning adhesive molecules, e.g., a 1 mg/ml solution of fibronectin or collagen I, on the surface of a channel. After the adsorption of these adhesive molecules onto the surface, the first layer of cell suspension, e.g., fibroblasts, is delivered and allowed to attach for 2 hours at 37° C. Then, a second layer of cells within a matrix solution, e.g., smooth muscle cells (SMC) in collagen, is transported through the microchannels. Because the first layer of cells are secured by adhesive molecules, the range of effective flow rates for delivering the second layer can be quite large (e.g., from 1 μl/min to 10 ml/min). For cells attached on channel surfaces without adhesive molecules, cells are more easily detached from the surface under the flow of a cell-matrix fluid. The third layer of cells is then attached on top of the second cell-matrix layer. The second cell-matrix serves as an adhesion layer for both top and bottom cell-matrix layers.

Strategy 2 is designed for facilitating the multilayer patterning of different types of cells as well as matrices. This strategy starts from the APTES-GA-activated glass/silicone slides. The microfluidic stamp is placed on top of the modified slides with the features facing down. As described earlier, the first layer of cell-matrix is patterned using simple injection, without the need to closely control the flow rate. After the polymerization of the first layer, culture medium is supplied for culturing. Depending on the contraction curve of a cell-matrix under the particular conditions, and the requirements for the thickness of the first layer, the culturing time is controlled. A contraction curve can be generated using microscopic channel measurements over time, as described above, and using the data to generate a contraction curve, as would be known in the art. When the time for culturing the first layer of cell-matrix is up, a new assembly of cell-matrix is prepared for the second layer and it should be injected using a syringe pump with strict control over the flow rate to avoid the elimination or disturbance of the first layer. The effective flow rate is dependent on many factors, such as effective channel height (original channel height minus the height of the first layer after contraction), channel width, mechanical properties of cell-matrix of the first layer, viscosity of the second layer fluids, and the like. The upper limit of the flow rate primarily depends on the viscosity of the second layer fluids and the mechanical properties of the cell-matrix of the first layer, while the lower flow rate limit primarily depends on the effective channel height and width. Moreover, the delivery temperature will influence both the polymerization speed of any biopolymer and the cell death rate. Typical biopolymers will begin polymerizing at temperatures above 10° C., and with increasing temperature, the rate of polymerization increases. Thus, if a cell-matrix material were delivered at a temperature above 10° C., biopolymer polymerization is also taken into account in determining a lower limit for the flow rate. A countervailing consideration is that most cells die quickly at temperature below 20° C. In the experiments reported herein, suitable delivery rates have been found to include rates of 5-10 μl/minutes, with the delivery process for a given layer being completed with a cell-biopolymer material at about room temperature (25° C.). Informed by the disclosure herein, one of ordinary skill in the art would be able to determine suitable flow rates for a given cell culture layer using no more than routine experimentation. After the second layer, the same methods for culture and re-injection can be repeated for the next upper layer. Theoretically, if the channel is high enough and the contraction of the matrices by the cells is great enough, the layer number inside the channel can always be increased. In our practice, we find it is practical to build, e.g., a three-layer structure of biomimetic vascular layers.

Biopolymer gels such as collagen are biphasic materials with a network of fibrils and interstitial solution [Barocas et al. J. Biomech. Etig. 119, 137 (1997)]. The fibrils form a sparse but highly entangled network that effectively resists shear and extension, but has little compressive strength. The shear rheology of collagen gels has been characterized previously [Barocas et al., J. Biomech. Eng. 117, 161 (1995)]. The effects of shear stress on the existing cell-matrix layer due to the flow of the next cell-matrix layer being delivered are examined by an imaging system. The working fluids are solutions of fluorescent-labeled cells within the matrices. A pre-cooled syringe with an ice jacket is used in delivering the cell-matrices. Syringe pump (Model-11, Harvard Apparatus Inc, MA) is used to drive the laminar flow in the main channels and to control the flow rate. Cells in the existing cell-matrix layer (first layer) are stained red using CMTMR in CellTrack series (Molecular Probe, OR) and cells being delivered in the next layer are stained green using CMFDA. Flow is visualized by a fluorescent microscope coupled to a CCD camera, which is connected to a microcomputer running image software (ImagePro Plus software). The displacement of the existing layer is measured from the series of images in the delivery process. The time interval between each frame is 0.12 seconds. Real-time video of the flow at different flow rates shows that there are different responses of the cell-gel under the shear stress. For microchannels that are 500 μm wide and 300 μm high, and cell-gel contraction time set at 6 hours, the cell-gel network remains undisturbed at 5 μl/min, minimally displaced at 10 μl/minute, highly displaced at 14 μl/minute, and dispersed away at 15 μl/minute. For microchannels 350 μm wide, the effective flow rate to deliver the 2^{nd} layer of cell-matrix is between 5 and 12 μl/minute. In the process of 2^{nd} layer delivery, the existing cell-matrix encounters shear stress from the fluid, and the gel is displaced at a certain flow rate. FIG. 5 shows the time-lapse video image sequences of the bottom layer under the shear stress of fluidic delivery of a second layer. The displacement of the gel lattice is measured through the movement of the CMTMR-labeled cell inside the matrix. For convenient measurement of the displacement, images are overlapped (FIG. 5a-c). Results show that the displacement of the gel slows down and finally stops after 3.6 seconds. After fluidic delivery of the cell-matrix, medium is added at the inlet and outlet of the channel to prevent dehydration. Then, samples are put into an incubator (37° C., supplied with 5% CO₂) for polymerization. The resulting shear stress is estimated to be around 30-80 dyne/cm² under the specified conditions, which did not result in appreciable displacement and provides guidance for maintaining shear stress forces within acceptable limits (no appreciable displacement of previous layers) in other situations (e.g., different cell types, different biopolymers). Calculation of shear stress is done either by calculation using the equation related to the channel dimensions and viscosity of fluid (as described herein) or by empirical observation using the equation based on the deformation of the existing layer as a stress-sensing layer. The equation for calculating shear stress based on deformation is: τ=Gu/h_{g}. This equation is used to determine the wall shear stress by using a highly deformed gel layer as the sensing element, where G is the shear modulus (for a collagen matrix, 155 dynes/cm²), hg the thickness of the gel, and u the displacement of the gel layer. For an image sequence that displays displacement or deformation, the value of u can be estimated. Wall shear stress reflects the mechanical property of a cell-matrix (e.g., fibroblast-collagen) assembly. The shear stress here is close to physiological shear stress level in the blood, which is about 10-40 dynes/cm² [Cooke et al., Proc. Natl. Acad. Sci. (USA) 100, 768-770, 2003].

Establishing a Cell Culture

A system for microscale cell culture is established by combining all of the above elements. Multiple cell types in matrices are patterned inside the microchannel. After establishing the structure, at least two culture modes are foreseen for cell culturing.

One mode is called “in-channel” culture, and the other is called “out-channel” culture. “In-channel” culture is implemented by keeping the cell-matrix inside the channel throughout the period of cell culture. The culture medium is supplied at the inlet and outlet of the channels. The cell culture is channel-bound in this mode. “Out-channel” culture, in contrast to “in-channel,” is implemented by removing at least part of the boundary (e.g., the microfluidic stamp) of the microchannels after multilayer patterning and leaving the cell-matrix unbound by the channels during the period of cell culture. After removing the microfluidic stamp (e.g., PDMS stamp), the arrays of layered cell-matrix are cultured in the medium directly. Unbound by the channel, extensive migration of cells in the “out-channel” mode is found. In this case, cells are spread to the spaces without matrices rather than remodeling and migrating inside the matrices. In contrast, the “in-channel” culture mode is more similar to the situation, e.g., in the in vivo vascular system where the growth of cells in matrices is bound by the vessel wall, and the migration of the cells is directed by signal transport among the layers. The support of the closed channel helps to limit cell migration in the horizontal plane and to keep the cells to remodel the “tissue” inside the matrix. Thus, it is beneficial for microscale layered “neotissues” to maintain their patterned position and to start their remodeling process in the patterned area. Because the migration and remodeling process is confined inside matrices, the cell-cell interactions and cell-matrix interactions are also more similar to those in the tissues. Either culture mode may be used, however, depending on the application. For example, to model “neo-vascular tissue,” the “in-channel” culture is preferred; however, to model the sprouting process of angiogenesis, and to study the directed migration of HUVECs out of the vessels, the “out-channel” culture mode may be applied instead.

Building Layered Structures

A vascular in vitro model was built as a prototype of the microsystem to confirm that the new technique and the resulting microsystem performed as expected.

The vessel wall represents a highly plastic 3-D structure with a unique adaptive capacity to face changes in blood pressure, flow, and shear stresses taking place during development and in some vascular pathologies. In the vascular system, the tunica (layer) intima comprises the endothelium, basement membrane, and connective tissue. The intima is surrounded by a layer of muscular tissue known as the tunica media, with SMCs as the primary cellular constituent. The outermost layer of the vessel is connective tissue, known as the tunica adventitia. The medial layer exhibits the greatest variation throughout the circulatory system, reflecting the differences in pressure, volume, compliance, and function.

This functional behavior is due to the specific assembling of cellular and extracellular components into three distinct tunicae or layers. This tissue compartmentalization inherently gives rise to some structural-functional interfaces, the integrity of which allows for the maintenance of arterial wall homeostasis. Pathological stimuli can also target all wall layers and interfaces simultaneously or one layer/interface primarily [Schwartz et al., Circ Res. 77, 445465, 1995]. The vascular ECM is a complex mixture of collagens, elastic laminin, and basement membrane. ECMs not only serve as mechanical support in vitro, they also play important roles in cell function by providing adhesive/functional binding sites for cells, and serve as the reservoir for growth factors. The adventitia layer in vivo is made of fibroblasts and connective tissues, in which collagen I is the major component, and the medial layer is mainly composed of SMC, laminin and basement membrane. The basement membrane is comprised of structural proteins, such as collagen IV, as well as adhesive proteins, such as laminin and fibronectin.

Based on these facts, we designed a biomimetic two-layer “in-channel” culture by using fibroblasts within a collagen I biopolymer lattice as a “neo-adventitia” layer, and using SMC within various types of matrices as a “neo-medial” layer. The matrices used with SMCs were explored for their impact on the maintenance of layered structure, cell-cell interaction, and matrix remodeling. The endothelium layer was formed by microfluidic delivery of a HUVEC suspension on top of an SMC-matrix after SMCs were cultured for one day. As the endothelial cell monolayer coating the walls of blood vessels, HUVECs were delivered without a biopolymer, demonstrating that the top layer of a multilayered microculture according to the invention need not contain a polymerized matrix. That is, following preparation of any underlying layer(s) in a multilayered microculture, the top layer may be applied as cells with, or without, a polymerizable biopolymer. In the case of the exemplified endothelial cell-containing microculture, the endothelial cells are able to attach to the cell-gel matrix (i.e., the second biolayer from the top of the microculture) with which such cells come into contact upon microfluidic delivery.

Cell Culture

Human lung fibroblasts (IMR-90, ATCC) were cultured in MEM (ATCC) containing 10% FBS. Human umbilical vein smooth muscle cells (HUVSMC, ATCC) were cultured in F-12K (ATCC) containing 1 mg/ml endothelial cell growth supply (ECGS), 0.1 mg/ml heparin, and 10% FBS. Human umbilical vein endothelial cells (HUVECs, Biowhittaker) were maintained in EGM-2 (Biowhittaker). Cells were expanded and large frozen stocks were prepared from the 2^{nd} passage. For experiments, all cell cultures were initiated from frozen stocks, and cells from the 3^{rd} to 7^{th} passages were used.

Multilayer Structure of Cells

Different cell types were used in the multilayer system built inside the channels and, thus, it was important to be able to differentiate them in the system. CellTracker probes (Molecular Probes) are suitable for long-term tracing of living cells and were used for this purpose. Fixation for confocal microscopy was carried out by simply immersing samples in a solution of 4% paraformaldehyde in PBS for at least 12 hours, and rinsed with PBS. In order to prevent changes of the structure in the solution before characterization, the PDMS template was not removed from the substrate (i.e., the slide chip) throughout all processes. Because of the good optical properties of PDMS, confocal images were obtained without problems of quality. Zeiss LSCM 510 or Zeiss Pascal confocal microscopy was used in characterization. The image stack was a series of images of successive optical sections with a Z-step (i.e., the step, or movement, of the z-motor, which determines the distance in the z-dimension between two consecutive image slices) set around 2.5 to 3.5 μm. The lower the Z-step, the higher the resolution for 3-D reconstruction images. The working distance for the laser-scanning confocal microscopy (LSCM) is 130 μM. Some samples exceeded this limit and, therefore, multiple times of scanning on the same location were required. The misalignment of image stacks in this situation should be avoided. After obtaining the image stacks, LSCM built-in 3-D reconstruction software or MetaMorph imaging software was used to obtain 3-D images.

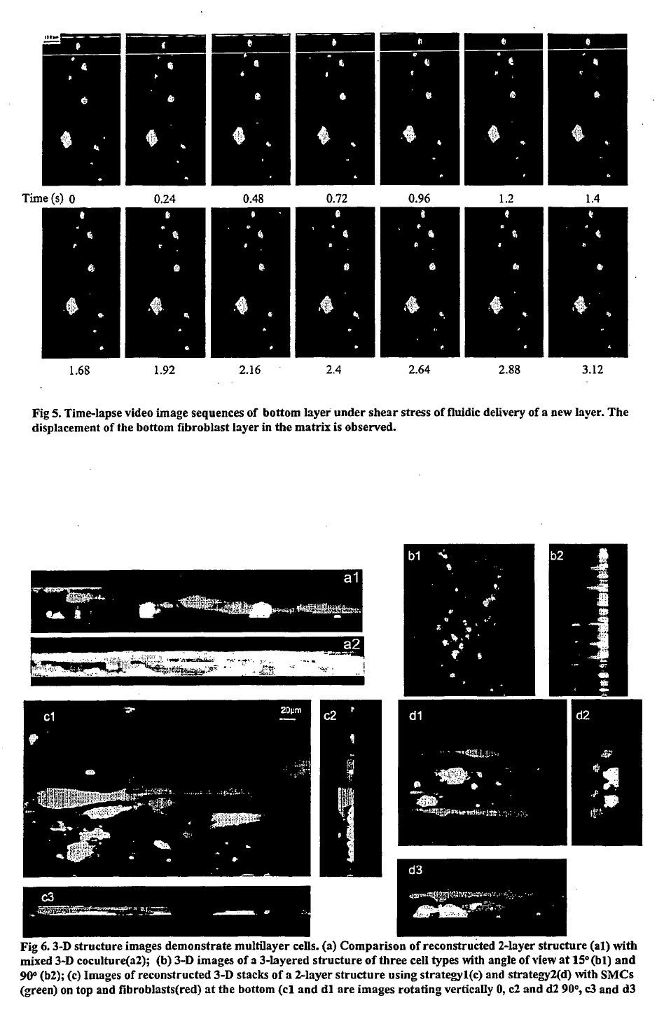

The 3-D structures established inside the microchannel were characterized by viewing the layered cells using confocal microscopy, as well as by viewing the microstructure of materials using SEM (scanning electron microscopy). Three cell types, endothelial cells, smooth muscle cells and fibroblasts were fluorescently labeled with different colors (red, green and blue) of CellTrack probes. Two-layer and three-layer cell-matrix structures inside the microchannels were prepared and imaged. FIG. 6a shows comparison between the configurations of layered co-culture of two cell types and the mixed co-culture. The layered culture is more organized. FIG. 6b shows the 3-layered structure of three cell types obtained from a fluorescent microscope. FIG. 6c-d shows 3-D images of the layered structure established using strategies 1 and 2. The fibroblasts are more spread on the surface of the substrate than in the gel, and the thickness of the bottom layer is about 5 Jim compared to 20 pim in strategy 2. Strategy 2 is preferred over strategy 1 because it allows one to control the thickness of the each layer by adjusting factors that change the cell-gel interactions. It is known that cell concentration, gel concentration and components, and cell-gel interaction time all influence the contraction percentage of cell-gel and, therefore, the thickness of a cell-gel layer can be controlled by them. Typically, the higher the cell concentration, the higher the gel concentration, and the longer the cell-gel interaction time, the more contraction that cell-gel biolayer will undergo. Moreover, cells inside the gel for strategy 2 exhibited 3-D differentiation rather than the 2-D spreading characteristics seen in strategy 1.

Multilayer Structure of Biopolymer Matrices

Samples with collagen or collagen-chitosan gels with different proportions were fixed, dehydrated, and dried using the following methods. After rinsing with PBS, gels were fixed with 2.5% glutaraldehyde in 0.1M cacodylate buffer (pH=4) for 1 hour at room temperature. The 0.1M cacodylate buffer was diluted from 0.2 M cacodylate buffer purchased from Electron Microscopy Sciences and pH was adjusted using 1M hydrochloric acid. The specimens were washed three time with cacodylate buffer prior to dehydration through a series of graded alcohol solutions (diluted from 100% pure ethanol, Sigma), starting at ethanol concentration of 50% to 70%, 80%, 90%, and, lastly, 100%, with 4 minutes in each solution. Specimens were then put into hexamethyldisilazane (HMDS, Electron Microscopy Sciences) for 15 minutes. This was followed by transferring samples to a dry well and air-drying for about 15 minutes. Matrix pieces were mounted and some silver paint was added to the edges of the specimen. The resulting samples were sputter-coated and examined using scanning electron microscopy. JEOL JSM-6320F and Hitachi S-3000N microscopes were used, with the former one providing higher resolution.

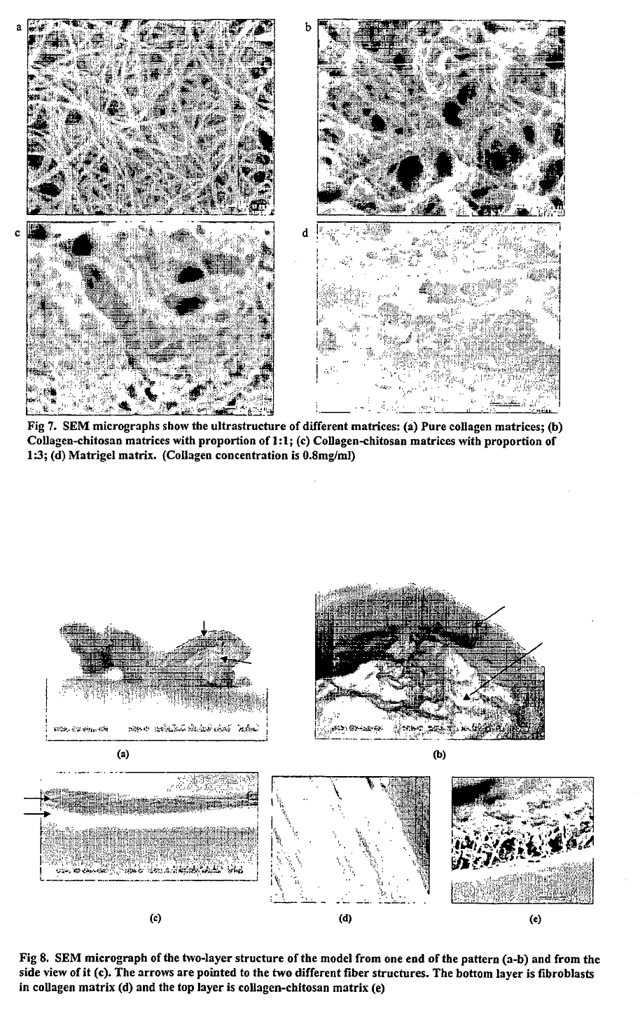

The advantage of multiple layers of cell-gel is not only to place different types of cells in layered structures, but also to make possible the creation of biomaterial gradients according to the mechanical and chemical requirements of the native tissue. This may be important to create biomimetic tissues because the layered structure of most tissues varies in terms of cellular composition and biochemical/mechanical properties. To illustrate this layered structure, collagen matrix and collagen-chitosan matrix were used. As shown in the FIG. 7, these two types of matrices are different in the microstructures of their fiber network. The figures show that when chitosan was introduced into the collagen solution, the formation of collagen fibers was not perturbed by the tiny chitosan clumps, because the diameter of fibers in pure collagen and those in gels with chitosan were almost the same. However, fibrous organization was significantly changed by chitosan, and pore sizes of gel matrices were greatly affected by different chitosan concentrations.

FIG. 8 shows scanning electron micrographs of the two-layer structure of the model, showing the structure from one end of the microstripe as well as the side view of it. FIG. 8 a-c was obtained from a Hitachi S-3000N microscope. The fiber network of the bottom collagen layer was observable under this microscope, but the top collagen-chitosan layer was more dense and unclear because the chitosan composite in the matrix greatly charged the electron beams. Thus, the sample was coated and viewed under a JEOL JSM-6320F microscope at higher magnifications. The fiber network was different from that of collagen alone (FIG. 8e). The bottom collagen layer was shining because of the laterally aligned fiber network, which is observed in FIG. 8d. The proportion of the two layers can be controlled by the contraction time of the first layer. In the example, the first layer was allowed to contract for about 16 hours, and the contraction curve in the figure reveals an approximately 50% contraction.

In conclusion, we introduced microfluidic methods to the construction of a 3-D, heterogeneous, cell-containing, multilayer structure inside microchannels. Using this approach, patterning of biological microstructures can be achieved not only on the surface (2-D), but also over the thickness of the construct (the third dimension). The “neotissue” formed of different types of cells and biopolymer components can be engineered to model inz vivo living systems.

### EXAMPLES

### Example 1

A microscale 3-layer microculturing structure for vascular tissue engineering was prepared. The multilayer system described above was used to model the vascular system, and the in-channel culture mode was used. The biomimetic system for vascular tissue engineering was investigated by separating the system into two parts: co-culture settings with a bilayer of “neo-adventitia” and “neo-media,” as well as co-culture settings with a bilayer of “neo-media” and endothelium.

Cell Migration Between Layers

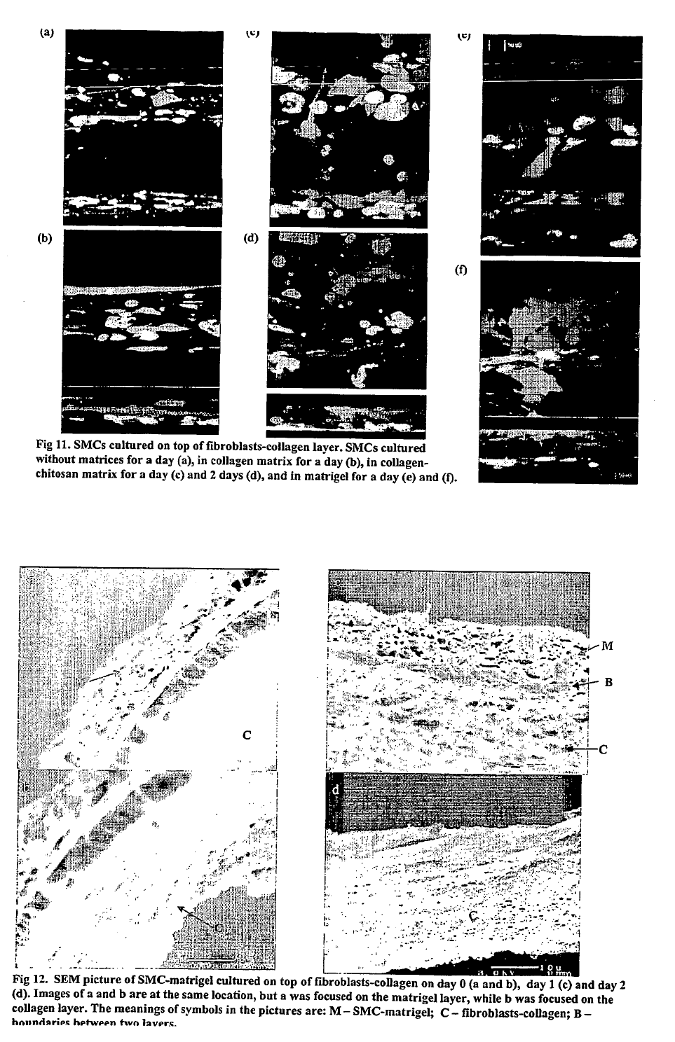

Our investigation revealed the differences among the various co-culture designs. FIG. 11 shows the 3-D reconstructed confocal images for different co-culture settings. For co-cultures with an SMC-collagen layer directly attached on top of the fibroblast-collagen layer, SMC (labeled with green fluorescence) invaded into and migrated inside the fibroblast layer easily, so that the two-layered structure could not be maintained in this situation (FIG. 11a). This is a typical iin vitro model currently used for co-cultures. For SMCs patterned within collagen matrices on top of fibroblasts-collagen, the two-layered structure was still visible after culturing for a day; however, the lamipollar extensions of SMCs were extended to the bottom layer, which increased the interactions between the two types of cells, in addition to the interactions at the border area (FIG. 11b). The yellow color in the confocal micrographs indicates the overlapping of red and green fluorescence. For SMCs patterned within collagen-chitosan matrices, limited migration or lamipollar extension of SMCs into the “neo-adventitia” layer was found after culturing for one day (FIG. 11c); however, after culturing for two days, the migration activity was increased (FIG. 1 id). Compared to all of the co-culture settings described above, SMCs patterned in matrigel matrix (reconstituted basement membrane) were demonstrated to have more control over the migration between the layered structures so that the cell-cell interaction between two types of cells was also better controlled. As shown in FIG. 11e-f, after culturing one or two days, SMCs remained in the matrigel and the extension of lamellipodium also existed just in the top layer. The direct SMC-fibroblast contact was very limited. Because the confocal microscope was limited to 130 μm for the 3-D stacks, the two-layered structure of matrigel-collagen exceeded this limit, and therefore multiple 3-D stacks had to be taken at the same location and merged to reconstitute the 3-D images.

In addition to the findings of different cell migration patterns in co-culture settings, differences in cell alignment inside the different matrices were observed. Elongated fibroblasts in collagen were found to align themselves in the direction of the channel axis. Cells in collagen-chitosan and matrigel were less aligned in any one direction.

Except in pathological or other abnormal conditions, the structure of a normal vessel in the adult is comparatively stable, with little migration of SMCs from medial to adventitia layers. The interactions of two types of vascular cells in vivo is limited to those lying on the border of the two layers, or even less (because of the separation of the cells by another matrix layer—laminin), and the cell interactions are quite different from the mixed culture that is commonly used for studying cell-cell interactions. In mixed co-cultures, the interactions between fibroblasts and SMCs, for example, are extensive and random, with interactions at different angles and places. In contrast to that, in the layered co-cultures, the cell-cell interactions or cell-cell contact was limited and well controlled. Among those co-culture settings, co-cultures with SMCs in matrigel on top of fibroblasts in collagen were found to be the best one in terms of retaining structures. The differences in cell adhesion to various matrices may help to explain the results. These findings suggest that cells are capable of migrating from their native ECM onto an adjacent matrix scaffold, and the zone of that is not obvious. Matrigel is a reconstituted basement membrane, the matrix type that surrounds SMCs in vivo. Thus, we found that the more in vivo-like design produces better biological effects on cells and structures, insofar as the cells and structures more closely mimic the in vivo condition. Furthermore, the in vitro layered structure was not only important to the control of cell-cell interactions, it also plays an important role in forming structure and positioning cells to facilitate the remodeling process.

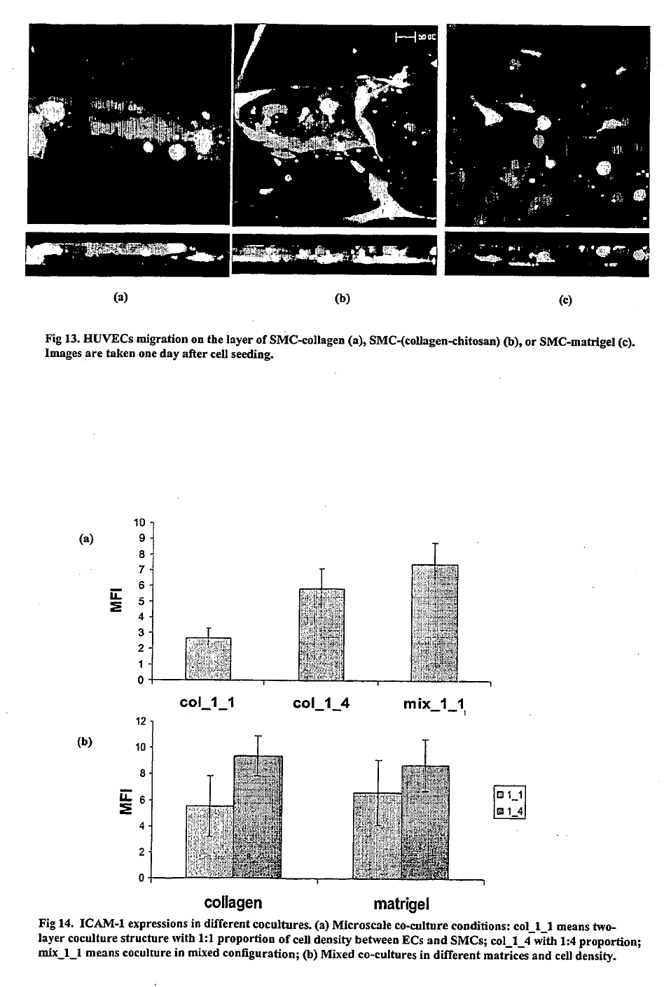

The biomimetic “neo-medial” layer was also set up with different matrices: collagen, collagen-chitosan and matrigel. The endothelium layer was formed by microfluidic delivery of HUVEC suspensions on top of SMC-matrix after SMCs were cultured for one day. Because of the difference in the contraction rate of the matrices by SMCs, the concentration of the HUVEC suspension should be adjusted to let the final cell density be equivalent in different co-culture settings. This experimental setup is similar to those widely used for an invasion model comprising collagen. FIG. 13 demonstrates the migration of HUVECs into the assembly of SMCs and the different matrices after co-culturing for one day. Migration of HUVCs into the matrices of collagen and collagen-chitosan was observed. In contrast, the matrigel matrix seems to limit the progress of cell migration. Therefore, the best biointegrating interface for ECs, in terms of mimicking the in vivo condition, is their native basement membrane, which contributes to the maintenance of the architectural and functional integrity of the endothelium in vivo. According to current understanding of vascular structure, endothelial cells in the endothelium have very limited direct contact with smooth muscle cells. The ECM, basement membrane and laminin, occupies the most space between these two types of cells. Tissue damage may cause the two types of cells to have more and closer contact. Our results show that modulating the matrix components could affect cell differentiation processes. Additionally, the data indicate that controlling cell migration will provide control over direct cell-cell contact. This observation was confirmed by experiments conducted with co-culture microcultures involving “neo-adventitia” and “neo-media,” which is that the presence of matrigel in the “neo-medial” layer allowed better control over cell-cell interactions between two layers.

Tissue Remodeling

The SEM micrographs demonstrate the structure of matrices after experiencing the process of cell remodeling in bi-layer co-cultures. FIG. 12 demonstrates SEM pictures of a SMC-matrigel layer cultured on top of a fibroblast-collagen layer after culturing for 0 day, 1 day and 2 days. In all of these pictures, two layers with different fiber structures were found. On day 0, the two structures seem to be just piled together with the matrigel layer simply lying on top of the collagen layer (FIG. 12a-b). There is no intermingling between the fiber networks. After culturing for one day, the two layers still maintain their own fiber structure, but a denser fiber network between the layers is found (FIG. 12c). In addition, these micrographs demonstrate the formation of a distinct boundary between the layers of matrigel and collagen. Finally, after culturing for two days, the two layers still exhibit distinct fiber structures (FIG. 12d); however, the structures in both layers are quite different from those on day 0 and day 1. This may result from the remodeling work by both SMCs and fibroblasts.

The results from the bilayer co-culture with a SMC-matrigel layer on top of a fibroblast-collagen layer demonstrated morphological and cellular boundaries between the co-cultured cells. These characteristics are similar to those features found in the formation of vessels in2 vivo. Formation of the great vessels in vivo involves multiple cell types that rearrange themselves at the correct space in the body to realize a precise morphogenetic plan. Similarly, in our in vitro model, we set up a pre-pattern on which the vessels can be built to achieve directed vessel formation. Our multilayer patterning approach created the positional restrictions for the cells at different layers. Likely because of the different components in the matrices used in the two-layer microculture, the layers set up boundaries between the two neighboring “neo-tissues” during the remodeling process and controlled the spatiotemporal migration of cells.

### Example 2

Structure-function relationships in neomedia and endothelial bilayers were investigated.

Immunofluorescence

Adhesion molecule expression was measured by means of fluorescence microscopy applying Image-pro plus software analysis. Mean fluorescence intensities (MI) for HUVECs and SMCs in co-culture settings were compared to the MFI of unstimulated cells. After removing the microchannel stamp from the slides, the slides were washed with PBS (or HBSS) three times. Then, serum PBS (10% serum in PBS, SPBS) was added on top of the slides. After incubating for 30 minutes at room temperature with gentle shaking, the slides were washed with PBS three times, for 5 minutes every time. Then, FITC-conjugated mouse monoclonal antibody (Sigma) in SPBS (diluted 1:100) was applied to the cell patterns on the slides. Slides were again incubated for 60 minutes at room temperature with gentle shaking. Finally, slides were washed with PBS three times, and observed using fluorescence microscopy.

Nuclear Staining

Slides were washed with PBS and fixed with 3.7% formaldehyde in PBS buffer for 15 minutes. Then, a Hoest 33342 (Molecular Probe, Eugene, Oreg.) solution was diluted 1:1000 in PBS, added onto the slides, and incubated for 30 minutes at room temperature. Finally, slides were rinsed with PBS for three times and observed under a fluorescence microscope. Cell concentration inside the 3-D matrices can be estimated from DNA content.

Cytoskeleton Actin Staining

The following protocol for staining actin with phalloidin was followed. After removing the microchannel template from the cell-pattern slides, cells were first fixed with 3.7% formaldehyde in PBS buffer for 15 minutes at room temperature with constant and gentle shaking. This was followed by aspirating the fixative and rinsing the slides with PBS for 3 times, for 4 minutes each time. Then, the cell membranes were permeabilized with cold acetone (−20° C.) for 5 minutes. Acetone was removed and slides were again rinsed 3 times. For staining, the phalloidin solution was prepared by diluting a stock solution 1: 200 in PBS immediately prior to staining and the diluted stain was added on top of the slides, about 600 μl per slide. Staining time was 30 minutes and, after that, slides were washed briefly with PBS and observed under a fluorescence microscope.

ICAM-1 expression

ICAM-1 is expressed in quiescent endothelium, but is up-regulated after cytokine stimulation in vitro and angiogenesis in vivo. The process of angiogenesis involves the processes of cell invasion, migration, and proliferation, and enhances the interaction of ECs with neighboring cells. ICAM-1 is also expressed on SMCs in embryogenesis as well as vascular diseases, such as atherosclerosis, restenosis and transplant vasculopathy in vivo. In vitro studies have characterized stimulatory and inhibitory factors that regulate ICAM-1 expression in cultured SMCs. There also is evidence for a biological function of ICAM-1 on SMCs for leukocyte accumulation and activation of mononuclear cells. Thus, ICAM-1 on SMCs may also contribute to the inflammatory reaction in the vascular wall. Because ICAM-1 is such a pivotal molecule in the physiology of both ECs and SMCs, we used ICAM-1 expression to further explore the significance of the structure of a cellular microenvironment on the cell biology in our studies. It was expected that there would be a significant correlation of ICAM-1 expression on cell patterns with the extent and the type of cell-cell contact. This expectation was confirmed by the results of experiments described herein.

FIG. 14 shows the quantitative comparison of ICAM-1 expression in the above-described different co-culture conditions. Results are expressed as the mean value ±standard deviation (STDEV), for n=11, p<0.01. Although not wishing to be bound by theory, the high STDEV might be due to the difference of ICAM-1 expression between the two types of cells. The mixed co-culture in collagen matrix shows a higher expression of ICAM-1 than the layered co-cultures. The cell proportion of SMCs to HUVECs also influences ICAM-1 expression. With increasing SMC cell number, ICAM-1 expression in the layered co-culture is also up-regulated. ICAM-1 expression in the layered co-culture in matrigel was too low to be detected. That may result from the limited SMC-HUVEC interactions in that co-culture setting, as seen from the section of cell migration. However, FIG. 14b shows that there is no significant difference in ICAM-1 expression among the mixed co-cultures in matrigel and collagen matrix with the same proportion of two cell types. Therefore, the configuration rather than the matrix type influences ICAM-1 expression on the co-cultured cells.

The properties of matrices will influence cellular configurations after the layered microculture stricture has been built and tissue culture efforts have begun. Further, “neo-tissue” configurations will also affect the way, and the degree, of cell-cell interactions between the same types of cells as well as the different types of cells. This may impact the cell biology and functions of such cells. In the experiments reported herein, the expression of a surface adhesion molecule (ICAM-1) changes with cellular configuration, which was determined by applying different types of matrices. Cell-cell interactions may involve interaction contact through direct contact between cells, as well as interactions through indirect contact, e.g., some cytokines or factors released from one cell and received by the other.

Cytoskeleton in the Bilayers

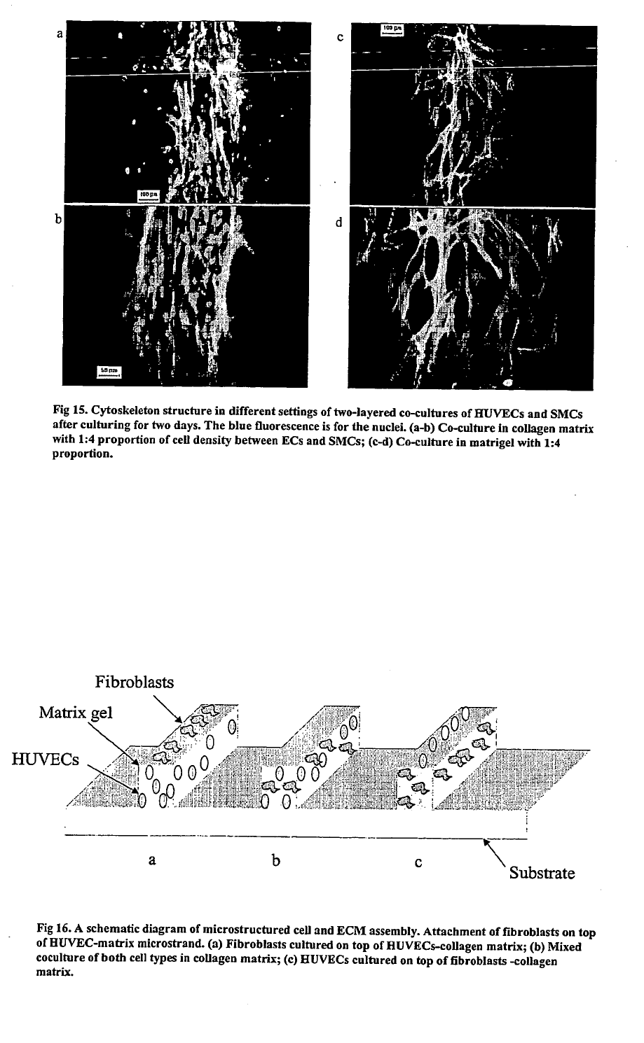

The orientation/alignment of co-cultured cells can be found by staining actin microfilaments in the cells. ECs co-cultured with SMCs were observed to be very elongated and were oriented randomly in a wavy pattern for the random co-cultures. However, the situation was not the same in the channel-bound co-cultures. FIG. 15 demonstrates the cytoskeleton structure (actin filaments) in different co-culture settings after culturing for 2 days. FIG. 15 shows that there was a significant difference between multilayer co-cultures in collagen matrix and in matrigel. The actin filaments were organized into large filament in cells. There was more cohesion of the actin filament of two cell types in rnatrigel than in collagen. The actin filaments were elongated and aligned with the channel axis in collagen, while the filaments formed capillary-like networks in matrigel.

Implementation of the in vitro model aspect of the invention provides for the study of cell co-culture that better mimics the in vivo environment than the conventional co-culture models in collagen gels. Building up microscale hierarchical structures of multiple cell types is important for tissue engineering. Use of a bioengineered substitute for the vascular system in vitro may facilitate the building of the system. Regeneration of a microvessel using the methods and devices of the invention is expected. The model established here allows for the analysis of cell migration, cell-cell interaction and cell-matrix interaction and remodeling, all of which are essential issues for microvessel regeneration.

### Example 3

Directed Cell Migration

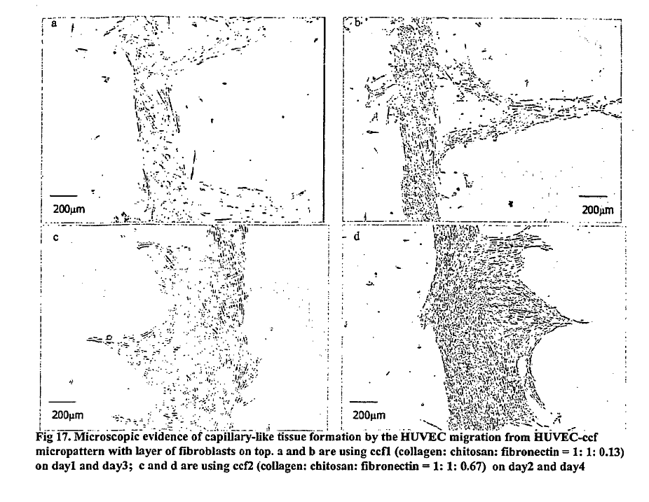

Flipping the position of cells in the matrix, wound healing models for cells can be established. As fibroblasts play an important role in vascular formation [Roy, 1997], they were chosen to co-culture with HUVECS. Sprout formation of ECs may be nonspecifically stimulated by nonendothelial cells possessing fibrinolytic activity; e.g., fibroblasts [Brown et al., Am J Pathol. 142(1), 273-83, 1993]. Such cells may support the migration and tubule formation of ECs by creation of a permissive matrix with formation of fibroblast-aligned channels, which might serve as guiding tracks for endothelial sprouts. Sprout formation of ECs was examined in the three types of co-culture configurations shown in FIG. 16. The “out-channel” culturing mode was used in all three co-cultures. However, HUVECs directed migration such that sprout formation was only found in the configuration shown in FIG. 16a, but not in the other two configurations. In the co-culture setting of FIG. 16a, HUVECs oriented along the channel direction and formed highly directed sprouting structures when co-cultured with fibroblasts on top. FIG. 17 shows co-cultured cells in collagen-chitosan-fibronectin matrices. The orientation of HUVECs in the stem of the micropatterns is either parallel to the axis of the channel or in the same direction as the sprouting structure. This behavior is matrix-related, and is observed with the collagen-chitosan-fibronectin matrix, but not the collagen or collagen-chitosan matrices. With cell proliferation over time, cell number in the stem increases and the stem becomes denser (FIG. 17b), but does not change in terms of size. Thus, matrices containing both cell types show better pattern integrity than matrices with HUVECs alone. Our studies suggest that fibroblasts are essential for HUVEC orientation (polarity), migration and proliferation, which is supported by the work on random co-cultures of fibroblasts and HUVECs [Black et al., FASEB J. 12, 1331-1340, 1998]. The HUVECs orientation guidance by fibroblast seeding may be caused indirectly by the orientation of collagen gels by the fibroblasts. The phenomenon of cell contact guidance in oriented collagen gels has been described previously [Guido et al., J. Cell Sci. 105, 317, 1993].

Vasculogenesis and angiogenesis involve the interaction of cells with other cells and their extracellular surroundings, These interactions are numerous and include direct cellular contacts, the production of local-acting mediators, and the release of distant-acting factors. The cells, ligands, receptors, and matrix are the key players in these processes. These cellular and molecular factors facilitate interactions that result in physiologically important phenomena such as cell adhesion, cell migration, tissue permeability, vascular patterning, and remodeling.

More generally, tissues of the body maintain a well-organized three-dimensional architecture. One of the challenges for the development of tissue engineering is the engineering of tissues that more accurately mimic the complex tissue microarchitecture found in vivo. Engineered tissue constructs require chemical and spatial control over cells to facilitate the assembly and organization of those cells in a functional structure.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features set forth herein and as follows in the scope of the appended claims.

1. A multilayer microculture comprising a plurality of three-dimensional non-fluid layers, wherein each layer comprises at least one cell type and a biopolymer selected from the group consisting of collagen, chitosan, fibronectin, matrigel, fibrin, and mixtures thereof, and wherein each layer comprises a width less than one millimeter.

2. The microculture according to claim 1 wherein each layer comprises a distinct cell type.

3. The microculture according to claim 1 wherein at least one layer comprises a plurality of cell types.

4. The microculture according to claim 1 wherein at least one layer is attached to an optically transparent support.

5. The microculture according to claim 1 wherein said layers comprises a first layer that is immobilized and wherein said first layer is resistant to a shear force associated with a 5 μl/min lateral flow of a cell-biopolymer fluid across the face of said first layer.

6. The microculture according to claim 1 wherein said microculture mimics a mammalian tissue.

7. The microculture according to claim 1 wherein said cell type is a non-contractile cell.

8. A method for producing a multilayer microculture comprising:
(a) introducing a first material comprising a first cell matrix compound and a first cell type to a microstructure by microfluidic delivery, wherein said material is introduced as a fluid;
(b) attaching said first material to at least one surface of said microstructure;
(c) incubating said first material under conditions suitable for at least one component of said material to polymerize and for said material to contract in at least one dimension; and
(d) repeating step (a) with a second material comprising a second cell matrix compound and a second cell type;
(e) attaching said second material to said first material; and
(f) incubating said second material under conditions suitable for at least one component of said second material to polymerize, thereby producing a multilayer microculture.

9. The method according to claim 8 wherein said first cell type and said second cell type are the same.

10. The method according to claim 8 further comprising:
(a) incubating said second material under conditions suitable for said second material to contract; and
(b) preparing a third layer of microculture by repeating steps (d)-(f) of claim 8.

11. The method according to claim 8 wherein said microstructure comprises a plurality of microchannels and at least one microfluidic aperture.

12. The method according to claim 8 wherein said material is a cell culture medium.

13. The method according to claim 8 wherein said conditions comprise time sufficient for said material to become a gel.

14. The method according to claim 8 further comprising attaching said material to said support.

15. The method according to claim 14 wherein said support is a derivatized glass.

16. The method according to claim 15 wherein said glass is derivatized by the presence of amine groups.

17. The method according to claim 16 further comprising an aldehyde cross-linker attached to at least one of said amino groups.

18-19. (canceled)

20. A method for identifying a modulator of tissue development comprising:
(a) incorporating a candidate modulator of tissue development into at least one layer of a microculture according to claim 1;
(b) incubating said microculture; and
(c) measuring the tissue development in the presence of said candidate modulator relative to the tissue development in the absence of said candidate modulator, wherein a difference in response relative to a microculture lacking said candidate modulator identifies a modulator of tissue development.

21-25. (canceled)

26. The method according to claim 20 further comprising attaching said microculture to a solid support.

27. The method according to claim 20 wherein each layer of said microculture comprises a distinct cell type.

28. The method according to claim 20 where at least one layer of said microculture comprises a plurality of cell types.
